(19) United States
(12) Patent Application Publication
(10) Pub. No.: US 2023/0010851 A1
(43) Pub. Date: Jan. 12, 2023

(54) Multi-layered Electrospun Heart Valve Leaflets

(71) Applicant: Xeltis AG, Zurich (CH)

(72) Inventors: Yonatan Gray, Paris (FR); Martijn Antonius Johannes Cox, Budel (NL)

(21) Appl. No.: 17/784,753

(22) PCT Filed: Dec. 18, 2020
(86) PCT No.: PCT/EP2020/086985

§ 371 Date: Jun. 13, 2022

Related U.S. Application Data
(60) Provisional application No. 62/951,530, filed on Dec. 20, 2019.
(60) Provisional application No. 62/951,501, filed on Dec. 20, 2019.

(51) Int. Cl. A61F 2/24

(52) U.S. Cl. CPC A61F 2/2412; A61F 2210/0076; A61F 2220/0075

(57) ABSTRACT

Multi-layered engineered designs are provided for heart valve leaflets. The multiple layer designs have significantly improved mobility of the leaflets, without reduced durability, compared to an ordinary single-layer design. In addition, a folded double layer design also showed highly reduced the risk of fraying.

### FIELD OF THE INVENTION

This invention relates to tissue engineering of heart valve leaflets.

### BACKGROUND OF THE INVENTION

Heart valves undergo tremendous stresses each heart beat while blood is being pumped through the cardiovascular system. Besides these stresses during each heartbeat, the heart valves also require significant durability lasting preferably a lifetime.

Prosthetic heart valves or engineered heart valves replace the natural heart valve in case of failure or injury to the natural heart valves. These prosthetic/engineered heart valves are single-layered heart valves and typically made from chemically fixated biological tissue (e.g. pericardium), yet they have limitations as they tend to heavily calcify long term and result in limited leaflet mobility and leaflet durability.

In some prosthetic/engineered heart valves materials are used to allow for Endogenous Tissue Restoration (ETR). The heart valve material would then be porous and bioabsorbable making the material capable for the heart valve to be absorbed by the body and replaced by natural tissue due to ingrowth of cells and nutrients into pores. However, the temporary nature of such an ETR-type heart valve may pose additional limitations to the durability of the heart valve.

The present invention addresses these problems of durability and mobility, and/or fraying of the leaflets, that originate from a single layered prosthetic/engineered heart valve and from heart valves that also desire ETR.

### SUMMARY OF THE INVENTION

The invention provides in one embodiment, a medical implant engineered as one or more leaflets of a heart valve. The leaflet has two electrospun layers which are attached only at the edges of the two electrospun layers allowing for maximum and independent movability of the one layer relative to the other layer in the leaflet. The leaflet can be porous and bioabsorbable making the leaflet capable to be absorbed and replaced by natural tissue due to ingrowth of cells and nutrients into pores (ETR).

In another embodiment, the invention provides a medical implant engineered as a leaflet of a heart valve. In this case, the heart valve defines a base and a free edge. The leaflet has a single electrospun layer folded over itself to create and have a bi-layered design for the leaflet such that the free edge of the heart valve is the folded edge of the leaflet. Only the remaining un-folded edges of the electrospun layer are attached and incorporated to the base of the heart valve allowing for maximum and independent movability of the one layer relative to the other layer in the leaflet. Like the first embodiment, the leaflet can be porous and bioabsorbable making the leaflet capable to be absorbed and replaced by natural tissue due to ingrowth of cells and nutrients into pores (ETR).

The double layer design has a significantly improved mobility of the leaflets, without reduced durability, compared to an ordinary single-layer design. In addition, the folded double layer design also highly reduced the risk of fraying.

In yet another embodiment, the invention provides a medical implant engineered as a leaflet of a heart valve. In this case, a middle layer in between the two electrospun layers. The middle layer in one example could remain independently movable from the two electrospun layers, and in another example the middle layer could be adhered to the two electrospun layers.

In yet another embodiment, the invention provides a medical implant engineered as a leaflet of a heart valve. In this case, a middle layer is in between the bi-layered design. The middle layer in one example could remain independently movable from bi-layered design, and in another example the middle layer could be adhered to the bi-layered design.

The middle layer could be varied to be a: (i) porous and bioabsorbable making the middle layer capable to be absorbed and replaced by natural tissue due to ingrowth of cells and nutrients into pores, (ii) an electrospun layer which is porous and bioabsorbable making the middle layer capable to be absorbed and replaced by natural tissue due to ingrowth of cells and nutrients into pores, or (iii) not bioabsorbable and capable to be permeated with natural tissue due to ingrowth of cells and nutrients into pores.

The middle layer and the two electrospun layers are electrospun with the same material or different materials. In another embodiment, each of the two electrospun layers are electrospun with the same material or different materials. Similarly, the middle layer and the bi-layered design are electrospun with the same material or different materials.

The tri layer design also has a significantly improved mobility of the leaflets, without reduced durability, compared to an ordinary single-layer design. In addition, the folded double layer design for this tri layer design also highly reduced the risk of fraying.

In still another embodiment, the invention provides a medical implant engineered as a leaflet of a heart valve. In this case, the leaflet has two electrospun outer layers and a middle layer, whereby the middle layer is laminated between the two outer layers and not independently movable from the two electrospun outer layers. The middle layer could be varied to be a: (i) porous and bioabsorbable making the middle layer capable to be absorbed and replaced by natural tissue due to ingrowth of cells and nutrients into pores, (ii) an electrospun layer which is porous and bioabsorbable making the middle layer capable to be absorbed and replaced by natural tissue due to ingrowth of cells and nutrients into pores, or (iii) not bioabsorbable and capable to be permeated with natural tissue due to ingrowth of cells and nutrients into pores. The middle layer and the two electrospun layers are electrospun with the same material or different materials. Also, each of the two electrospun layers are electrospun with the same material or different materials.

### BRIEF DESCRIPTION OF THE DRAWINGS

FIG. 1 shows according to an exemplary embodiment of the invention electrospun fibers 120 deployed on a cylindrical metal mandrel 110.

FIG. 2 shows according to an exemplary embodiment of the invention the electrospun fibers 120 while still on the cylindrical metal mandrel 110 but now laser cut according to a laser-cut pattern as shown in FIG. 3. Curved edges 210 are referred to as the heart valve base (see also FIG. 6), and diamond shapes 220 are referred to as the shape commissures of the heart valve (see also FIG. 6).

FIG. 3 shows according to an exemplary embodiment of the invention a laser-cut pattern 300 to laser cut the electrospun fibers 120 while still on the cylindrical metal mandrel 110 (noted that this is a two-dimensional representation of a three-dimensional shape).

FIG. 4 shows according to an exemplary embodiment of the invention the laser-cut electrospun heart valve shape 400 removed from the cylindrical metal mandrel (noted that the shape in FIG. 4 is 90 degrees rotated relative to FIGS. 1-2). This shape 400 is then to be folded across the diamond shapes 220 along the line 510 as shown in FIG. 5.

FIG. 5 shows according to an exemplary embodiment of the invention the fold line 510 where the shape 400 of FIG. 4 is to be folded (noted that this is a two-dimensional representation of a three-dimensional shape). The fold-line becomes the free-edge 610 of the heart valve shape as shown in FIG. 6 once folded.

FIG. 6 shows according to an exemplary embodiment of the invention the folded heart valve shape 600 (noted that this is a two-dimensional representation of a three-dimensional shape). Curved edges 210 become the heart valve base 620 (see also FIG. 2), and diamond shapes 220 become the V-shaped commissures 630 of the heart valve (see also FIG. 2).

FIG. 7 shows according to an exemplary embodiment of the invention a cross-sectional view of the folded heart valve shape 600 with folded free-edge 610 and heart valve base 620. It is noted that this is two layers of the electrospun fibers folded at the fold-line 510 (FIGS. 5-6) where the fold has become the free-edge 610. In an alternate embodiment a heart valve leaflet can be engineered by having independent two electrospun layers which are then attached/ad only at the edges (similar as what is referred to as the free-folded-edge) of the two electrospun layers allowing for maximum and independent movability of the one layer relative to the other layer in the leaflet. Regarding the third design having a middle layer, the middle layer is placed in between the folded layers as a skilled artisan would readily appreciate (see also FIG. 12).

FIG. 8 shows according to an exemplary embodiment of the invention a three-dimensional view of an electrospun folded heart valve 800 resulting from the steps as shown in FIGS. 1-7 prior to suturing, gluing, stitching or generally adhering this heart valve to a heart valve frame.

FIG. 9 shows according to an exemplary embodiment of the invention an image of the assembly 900 of the electrospun folded heart valve, like 800, assembled to a heart valve base 910.

FIG. 10 shows according to an exemplary embodiment of the invention fully closed and open phases of prototypes of heart valve assemblies produced by to the method steps provided herein and captured via a high-speed camera during hydrodynamic test.

FIGS. 11-12 show according to an exemplary embodiment of the invention the electrospun layer thicknesses and heart leaflet shapes that have been tested (see also Tables 1-2).

FIG. 13 shows (left) an upper view on a tri-leaflet application in an aortic surgical valve, and (right) section view of a leaflet where (A) is the inner layer (a polymer as listed in the materials), (B) is the middle layer (PET woven sheet), (C) is the outer layer (a polymer as listed in the materials), and (D) is the folded free edge of the leaflet.

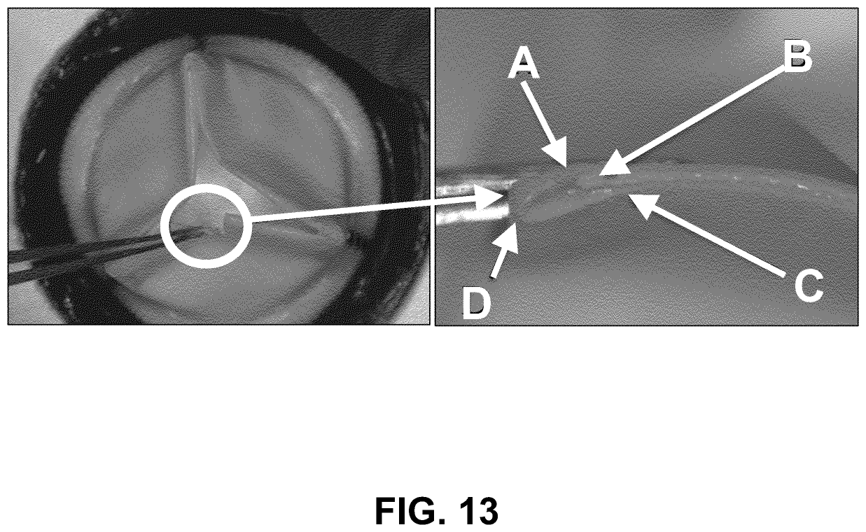

FIG. 14 shows an example of the woven structure as an example of the middle layer. Bottom image shows a Polyethylene Terephthalate (PET) woven sheet laser cut into a mesh of square holes as an embodiment of an intermediate durable layer. The top image shows a magnification of the PET micro-structure via a scanning electrons microscopy (SEM) (Top). The PET durable mesh is used as a middle layer with dedicated holes to allow chronic remodeling between inner and outer XP polymer layers.

### DETAILED DESCRIPTION

Embodiments of the present invention advances the art and overcomes problems like durability, mobility, tearing and/or fraying by introducing a multi-layered structure for the leaflets of the heart valve. A first design of a dual-layer is by having folded structure making a bi-layered structure, and a second design of a dual-layer is by adhering the edges of two independent electrospun layers at the free edge making a bi-layered structure. A third design is by having a dual-layered design with a layer in between the two layers of the dual-layered design.

In each design, the bi-layer leaflet of the heart valve is defined by two electrospun layers that preferably allow the maximum movement in between the layers to promote and ensure flexibility and mobility. In one embodiment, each layer in the bi-layer leaflet design could be produced from the same material, which is for example the materials as listed infra. In another embodiment, each layer in the bi-layer leaflet design could be produced from different materials. The latter is predominately true for the two independent electrospun layers that are adhered at the free edge.

The idea behind the bi-layer leaflet design is that two individual and independently moving layers in the design would be more flexible than a single layer leaflet if they would have the same overall thickness. The total thickness of the two layers combined in the bi-layer leaflet design is between 200-500 micrometers, and preferably 300-400 micrometers.

In the second design, it is preferred that the two layers are not adhered to each over the entire surface other allowing for the maximum movement independent from each other. To that effect, the two layers would be adhered or connected at the edges which comes to about 10-15% of the height of the heart valve leaflet. This could be accomplished by gluing, heat welding, or stitching the edges (or equivalent methods). As mentioned, in the first design, there is no need to adhere since that concept is accomplished by folding a sheet and only adhering the edges of the non-folder sides. The maximum movement ability between the two layers allows for more flexibility during bending, and this flexibility translates in better mobility and lower pressure gradients.

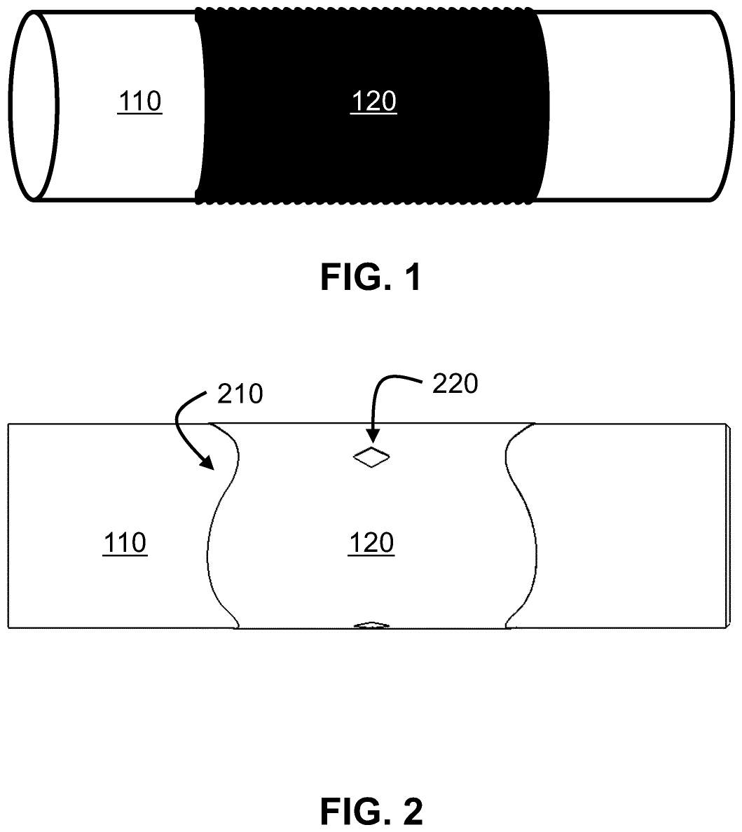

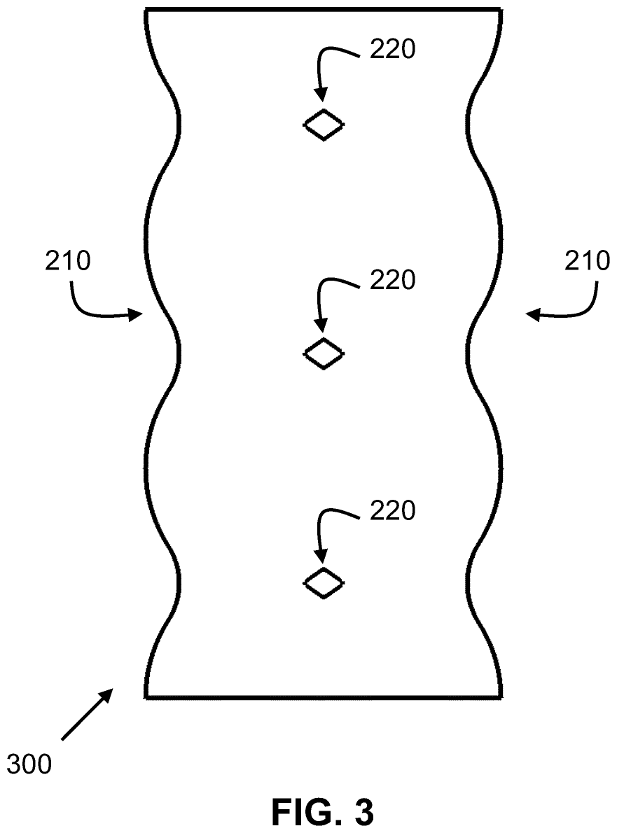

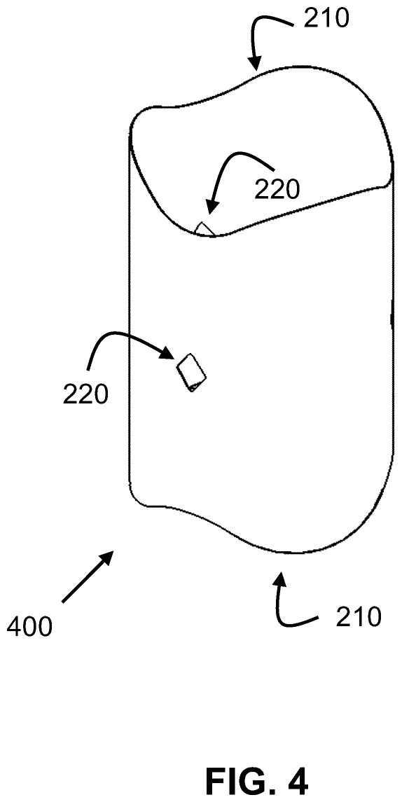

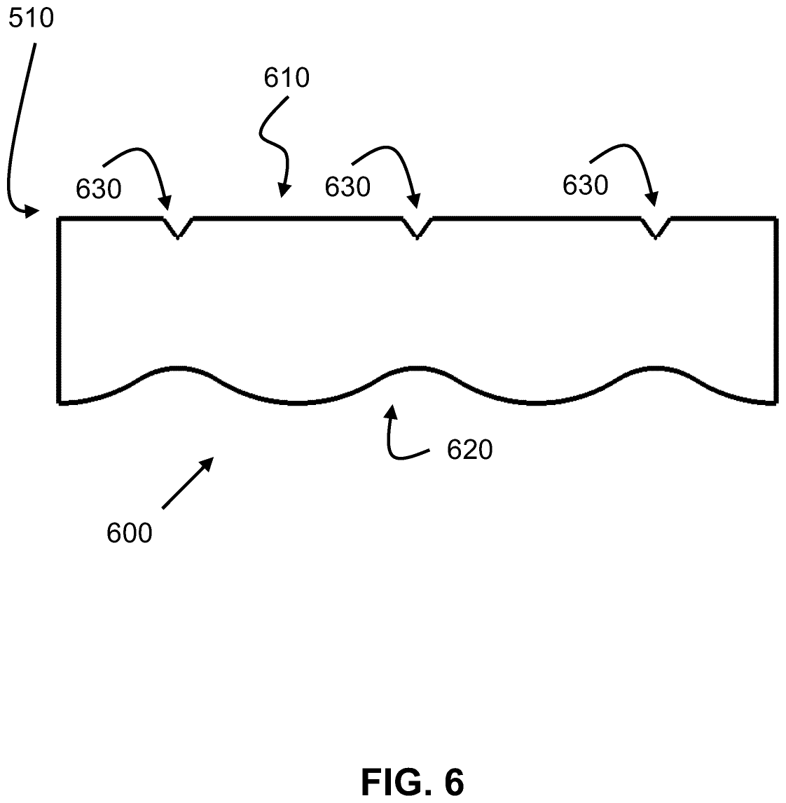

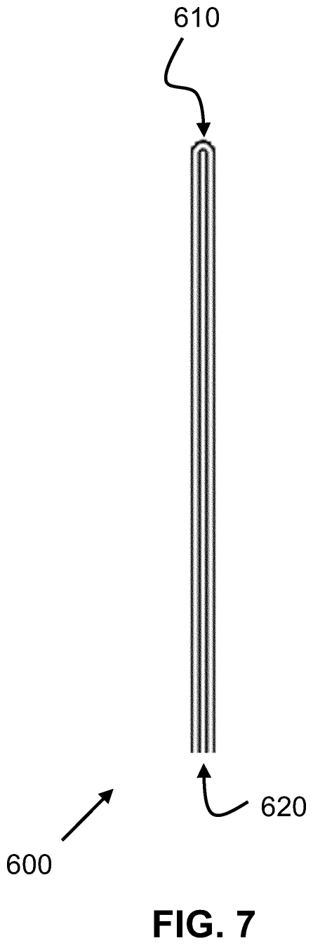

Directing to the first design, fibers are electrospun on a cylindrical metal mandrel 110 creating electrospun fibers 120 (FIG. 1). A list of useful fibers is provided towards the end of the description. Electrospun fibers 120 while still on the cylindrical metal mandrel 110 are then laser cut according to a laser-cut pattern 300 (FIGS. 2-3). Curved edges 210 are referred to as the heart valve base (see also FIG. 6), and diamond shapes 220 are referred to as the shape commissures of the heart valve (see also FIG. 6). Once laser-cut, the electrospun heart valve shape 400 is removed from the cylindrical metal mandrel. Shape 400 is then folded across the diamond shapes 220 along the line 510 (FIG. 5). In one example about three waves and about 3 diamonds are cut.

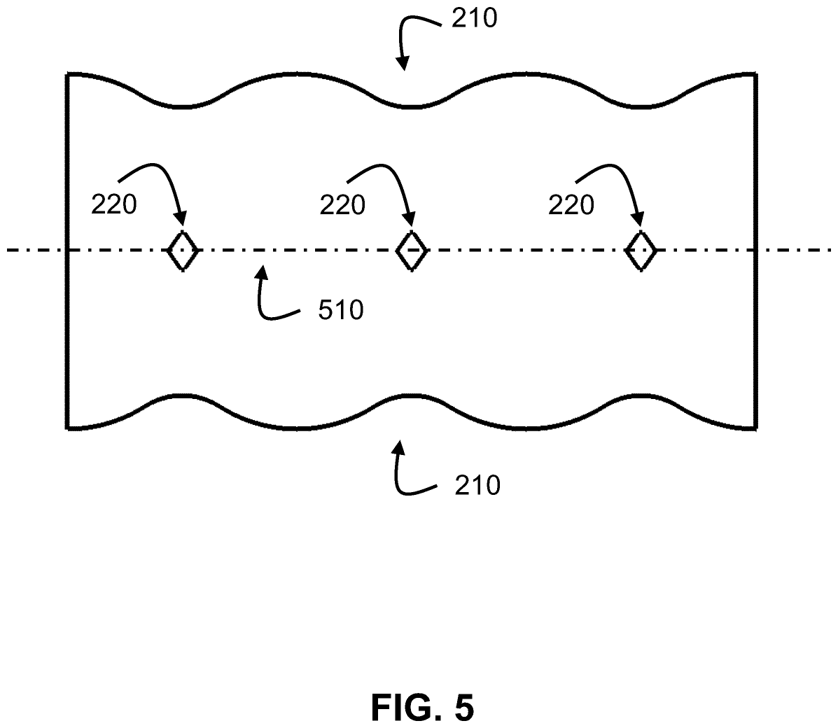

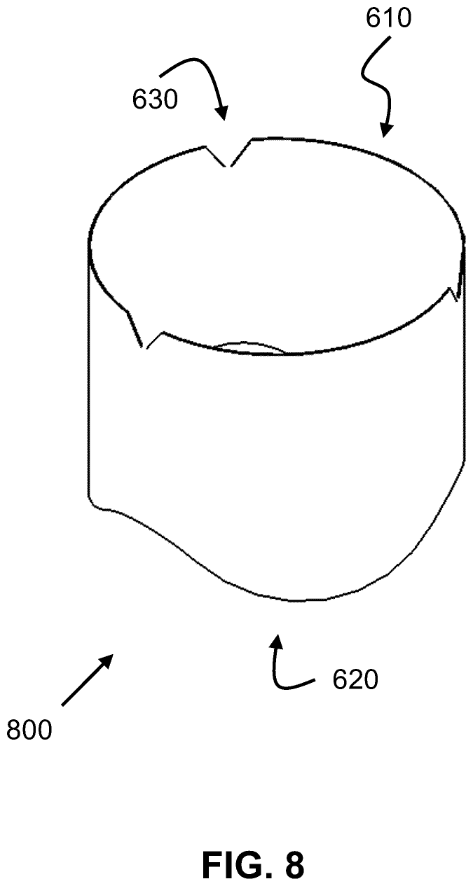

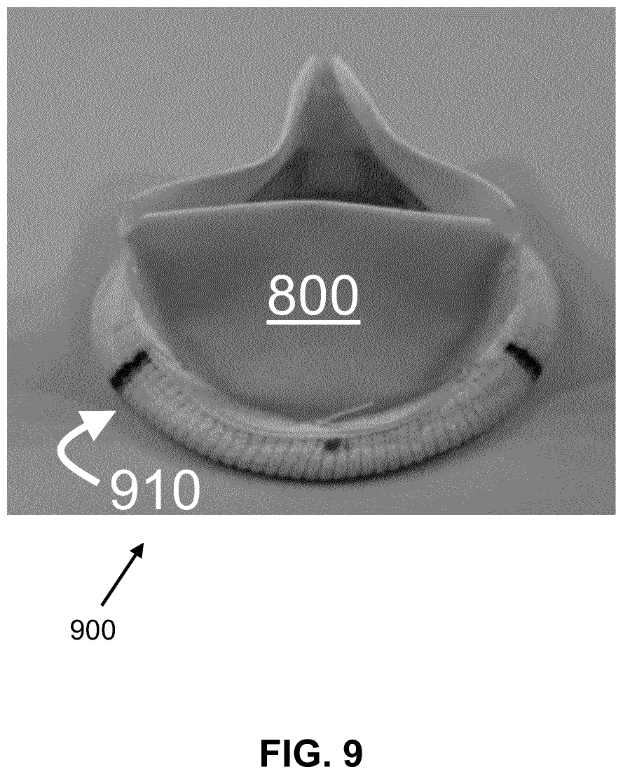

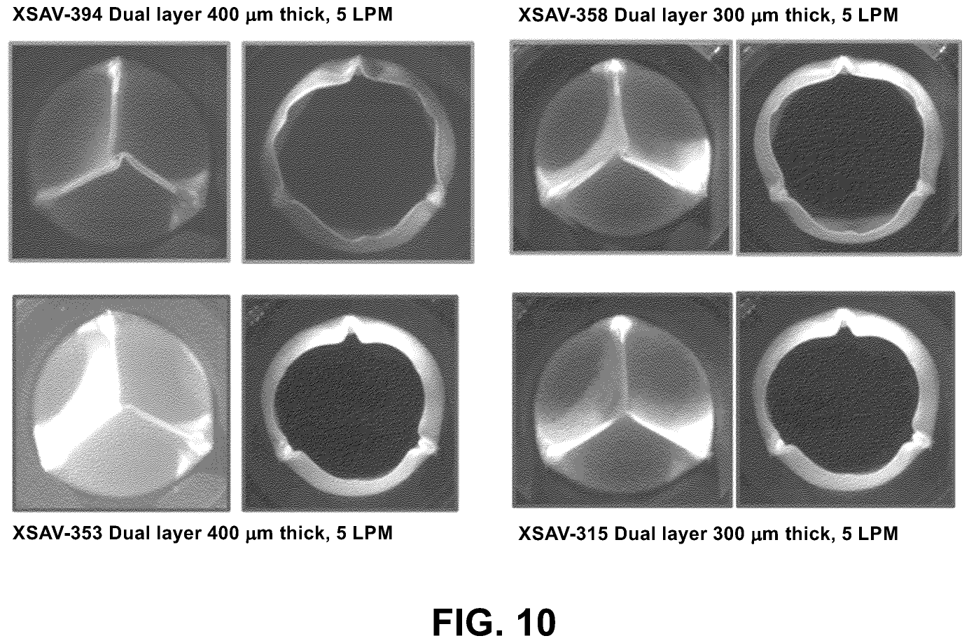

FIG. 5 shows according to an exemplary embodiment of the invention the fold line 510 where the shape 400 of FIG. 4 is to be folded (noted that this is a two-dimensional representation of a three-dimensional shape). Once folded, the fold-line becomes the free-edge 610 of the heart valve shape (FIG. 6). Once folded, curved edges 210 become the heart valve base 620 (see also FIG. 2), and diamond shapes 220 become the V-shaped commissures 630 of the heart valve (see also FIG. 2). FIG. 7 shows a cross-sectional view of the folded heart valve shape 600 with folded free-edge 610 and heart valve base 620. It is noted that this is two layers of the electrospun fibers folded at the fold-line 510 (FIGS. 5-6) where the fold has become the free-edge 610. As mentioned, in the alternate and second design a heart valve leaflet can be engineered by having independent two electrospun layers which are then attached/ad only at the edges (similar as what is referred to as the free-folded-edge) of the two electrospun layers allowing for maximum and independent movability of the one layer relative to the other layer in the leaflet. FIG. 8 shows the resulting electrospun folded heart valve 800 prior to suturing, gluing, stitching or generally adhering this heart valve to a heart valve frame. FIG. 9 shows an image of the assembly 900 of the electrospun folded heart valve, like 800, assembled to a heart valve base 910.

Each of the layers of the bi-layer leaflet (first and second) design can be characterized as follows:
Layer with micropores to allow Endogenous Tissue Regeneration (ETR). Pore size is about 1-100 micrometers.
Electrospun layer with a fiber diameter of about 4-10 micrometers in one example, and 1-20 micrometers in another example.
(Bio)absorbable layer, although not necessary the desirability depends on the application.
Made from a polymer as listed infra.
Thickness of the layer would typically be between 80-200 micrometers in one example, and 50-300 micrometers in another example.
The total thickness of the two layers combined in the bi-layer leaflet design is between 200-500 micrometers, and preferably 300-400 micrometers.

In the third design, a middle layer is placed in between the two layers of the dual-layered design. This means a middle layer added to the first design and a middle layer added to the second design as discussed above whereby the first design is by having folded structure making a bi-layered structure, and the second design by adhering the edges of two independent electrospun layers at the free edge making a bi-layered structure. In one option, the middle layer could be assembled in between such that only the remaining un-folded edges of the electrospun layer and the middle layer are attached allowing for maximum and independent movability of the layers relative to each other. In another option, the middle layer could maintain maximum and independent movability without any attachment.

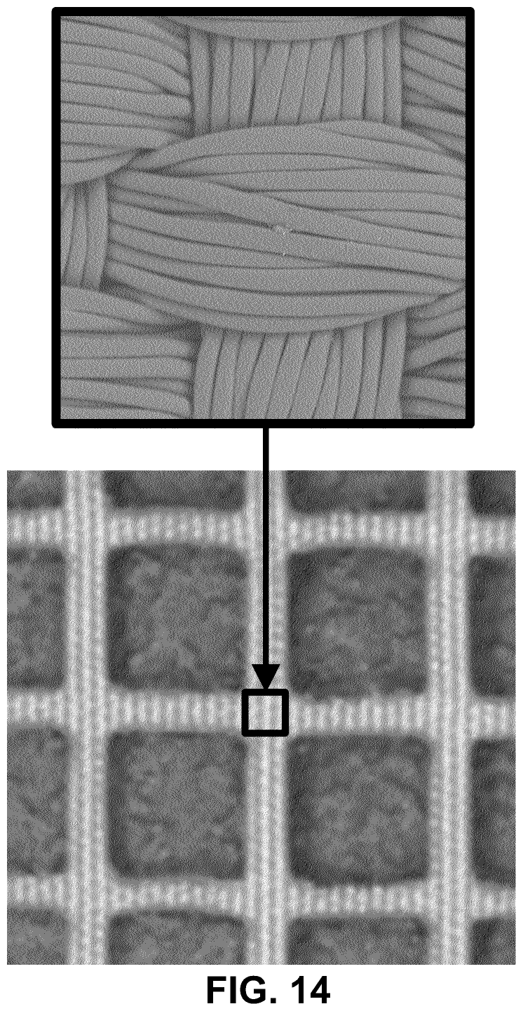

In one embodiment, the tri-layer leaflet design could be produced from the same material, which is for example the materials as listed infra. In another embodiment the tri-layer leaflet design could be produced from different materials. The key aspect of the middle layer relative to the outer layers is that the middle layer is of such material characteristics that they would increase the heart valve durability yet maintaining the mobility of the overall leaflets and therewith heart valve. In general, the middle layer is preferably more durable to improve the overall durability of the device even further. This could be achieved by choosing a different material or by a different texture (e.g. solid/woven/electrospun sheet with same or less porosity than the outer layers, see e.g. FIG. 14).

In general, the middle layer could be produced from a biodegradable or non-biodegradable material. Preferably it is produced in a way that degradation time is reduced compared to the outer layers. The middle layer could be ETR compatible or non-ETR compatible (then being incorporated into the created tissue). Preferably it will be ETR compatible when a biodegradable set-up is chosen. Possibly (ideally) the middle layer has micropores and/or micropores to allow ETR through its pores and/or to allow the outer layers to attach to each other through the electrospinning (the latter requires macropores and a relatively thin middle layer).

In an alternate embodiment, the middle layer could be produced by electrospinning. Electrospinning parameters should be adapted to create a higher strength (via variation of the fiber alignment, pore size, material, fiber thickness, etc.). In particular the fibers have a preferred orientation along the circumference of the leaflets similar to native leaflet orientation. Alternatively, the middle layer could be a film or a 3D printed structure.

In one embodiment, the material of the middle layer is biocompatible, durable and should either degrade very slow or even stay in the body. In one example, a thin woven fabric made of PET could be used. This layer has a different texture than electrospun outer layers where a tight bundle of filaments is constantly woven to increase strength. Permeability of the middle layer could be introduced by creating a patterned perforation of the support structure. The PET woven structure should be very durable to dynamic shear stress in comparison to the outer layer and its thickness is set to 60-100 microns. Architecture of woven structure (e.g. —number of filaments per bundle, yarn angle, permeability, patterned perforation, suture retention strength, etc.) can be tuned and tailored to a specific leaflet design (i.e. —to conform to a specific valve frame) (see e.g. FIG. 14).

In a second example a thin film 60-100 microns thick-casted of polymer solution (e.g. as described in the list infra) (not electrospun) could be used for a slow pace degradable middle layer with high durability potential. This middle layer will stabilize the leaflet while the ETR process takes place even later throughout the whole lifetime of the artificial valve in the body. Experiments have been performed and this tri-layer design shows an improved durability compared to simple double- or mono-layer designs. The middle layer would thereby absorb the stresses. Permeability of the middle layer could be introduced by creating a patterned perforation of the support structure.

In another example, the PET structure contains macro and/or micropores to allow ETR and/or a connection between the outer layers through electrospinning.

There are several methods to produce such multi-layer membranes or leaflets. According to one embodiment the outer layers are produced (e.g. by electrospinning). Then these layers are connected around the middle layer by heat welding, gluing or other methods.

The middle layer could also be described as a heart leaflet support structure embedded in between electrospun layers. The heart leaflet support structure could, without limitation:
Be sufficiently porous to allow ETR;
Sufficiently “open” to allow the inner and outer electrospun layer to laminate to each other.
May or may not be a non-degradable layer;
Be a woven (PET) mesh, suture wires, (metal e.g. nitinol) braided/knitted mesh; and/or
Potentially post-processed to generate local porosity/open cell structure.

Further, the middle layer could also be described as a heart leaflet support structure embedded in between electrospun layers. The heart leaflet support structure could, without limitation:
Be sufficiently porous to allow ETR;
Made of a film (same material or material class as electrospun outer layers);
Laminated to the electrospun layers, but it does not necessarily have to be sufficiently open to allow the electrospun layers to laminate to each other (i.e. they stick to the film already);

In the case where the free edge of the layers is adhered one could envision this to be about 10-15% of the height of the leaflet, which is about 1 mm. The rationale is to keep the free edge from delaminating and therefore to account for the about 10-15% of height which is (fully) laminated. The middle layer at its top will not reach the free edge, but about 1 mm below it. Yet in still another embodiment, for the dual-layer designs or the tri-layer design with the middle (heart leaflet support structure) lamination could be desired in certain applications versus allowing the layers to move freely with respect to each other.

### Testing

To assess and quantify in-vitro mobility of a tested valve, a commercially available pulse duplicator (A “Hydrodynamic tester” by BDC Labs, USA) was used. In this classical test apparatus, a valve is placed in a fluidized chamber with flow and pressure control. Compressed air combined with test fluid is utilized to mimic physiological conditions and blood vessels compliance. The hydrodynamic tester interface analyzes valve life performance, essential parameters are measured and quantified in an open phase of the valve (i.e. effective orifice area (EOA) [cm²], max and mean positive pressure difference (PPD) [mmHg]) and in a closed phase of the valve (i.e. closing volume (CV) [%], regurgitation fraction (RF) [%]).

The tester can accommodate various heart rates and cardiac outputs under desired pressures replicating range of physiological conditions of cardiovascular system.

To assess and quantify the improved durability of a multi-layer valve, a classic commercially available valve durability tester was used (An “accelerated wear tester” by BDC Labs, USA). In such a tester, the valve is placed in a pressure-controlled, sealed chamber while being submitted to fluid flow pushed through its leaflets via a linear motor and a piston. Piston location controls the opening and closing of the leaflets and frequency of the motor dictates the speed of a valve cycle (Hz). Dynamic pressure gradient over a closed valve is set to simulate physiological conditions. A valve tested can usually be run in frequency of 5-25 Hz, where for this application in consideration with a polymer and its viscoelastic properties, a frequency of 10 Hz was chosen. A cycle is counted only if 5% or more of the cycle duration while the valve is closed, is endured under a specific pressure gradient (i.e. —target pressure). In this application, target pressure was set at 100 mmHg.

Additional parameters are measured to assess valve mobility using a high-speed camera from various views. These parameters are not always standardized (according to ISO-5840), but could be valuable for characterization of valve dynamic performance, such as: leaflet to leaflet synchronization and overall symmetry in opening and closing phases, closed leaflet sagging, commissural deflection, etc. Some of these parameters are addressed mainly in product design development.

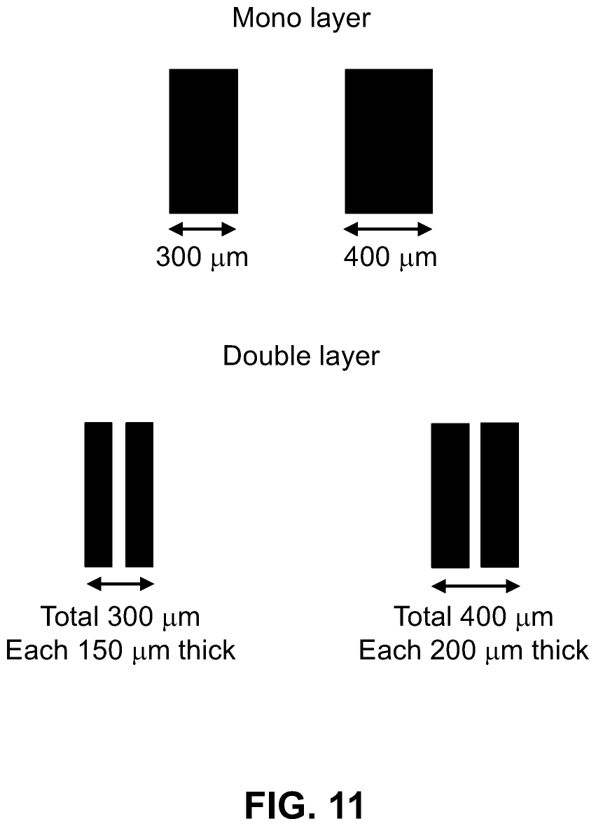

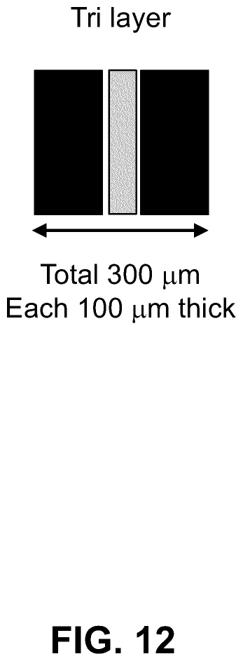

In FIG. 10 and Table 1 show results of in-vitro testing for mobility of bi-layer and single-layer valves of various leaflet thicknesses as shown in FIG. 11. FIG. 10 shows fully closed and open phases of prototypes of heart valve assemblies and captured via a high-speed camera during hydrodynamic test. The experiments show that a double layer design has a significantly improved mobility of the leaflets, without reduced durability, compared to an ordinary single-layer design. In addition, the folded double layer design also highly reduced the risk of fraying.

TABLE 1
Single-layer vs. Bi-layer leaflets in hydrodynamic tester. First
comparison shows that the embodiment of a 400 μm thick Bi-layer
leaflets (n = 2) is as mobile as Single-layer leaflets, while the
valve is in opening phase (effective orifice area and pressure gradient
are relatively the same) and inferior in closing phase (larger closing
volume leads to larger regurgitation fraction). Second comparison
suggests that the embodiment of a 300 μm thick Bi-layer leaflet
(n = 1) is more mobile than a Single-layer leaflet in opening phase
(higher effective orifice area, lower pressure gradient), as mobile
as a Single-layer leaflet in closing phase (similar closing volume).
Normotensive aortic conditions (CO = 5 LPM, P = 120/80 mmHg)

| | EOA | PPD | CV | RF |
|---|---|---|---|---|
| Valve | [cm²] | [mmHg] | [%] | [%] |
| XSAV-394, bi-layer, | 2.7 | 5.8 | 10.2 | 10.8 |
| 400 μm total thickness | | | | |
| XSAV-395, bi-layer, | 3.0 | 4.6 | 11.9 | 12.8 |
| 400 μm total thickness | | | | |
| XSAV-353, single-layer, | 2.6 | 6.3 | 5.8 | 5.9 |
| 400 μm total thickness | | | | |
| XSAV-355, single-layer, | 3.0 | 4.6 | 6.3 | 6.5 |
| 400 μm total thickness | | | | |
| XSAV-358, bi-layer, | 3.2 | 3.8 | 7.4 | 7.9 |
| 300 μm total thickness | | | | |
| XSAV-315, single-layer, | 2.5 | 6.6 | 7.1 | 11.5 |
| 300 μm total thickness | | | | |

TABLE 2
Results of a durability comparison between a tri-layer prototype
and three mono-layer prototypes showing an improvement of at least
one magnitude in cycles completed up to failure of the leaflets
where required pressure gradient was unable to be reached. In conclusion,
the tri-layer prototype had reached and passed the ISO standard
for heart valve durability of 400M cycles. This shows the potential
to pursue ETR in a heart valve with competitive durability compared
to the state of the art (biological tissue leaflets).

| | | Durability cycle | Failure |
|---|---|---|---|
| # | Type | count [Million] | mode |
| 1 | Tri-layer, 300 μm total | 405 | Commissural |
| | thickness, welded posts | | tear |
| 2 | Mono-layer 400 μm thick, | 10 (failed) | Commissural |
| | welded posts | | tear |
| 3 | Mono-layer 400 μm thick, | 27 (failed) | Commissural |
| | welded posts | | tear |
| 4 | Mono-layer 400 μm thick, | 34 (failed) | Commissural |
| | welded posts | | tear |

### Materials

The electrospun material referenced in herein may comprise the ureido-pyrimidinone (UPy) quadruple hydrogen-bonding motif (pioneered by Sijbesma (1997), Science 278, 1601-1604) and a polymer backbone, for example selected from the group of biodegradable polyesters, polyurethanes, polycarbonates, poly(orthoesters), polyphosphoesters, polyanhydrides, polyphosphazenes, polyhydroxyalkanoates, polyvinylalcohol, polypropylenefumarate. Examples of polyesters are polycaprolactone, poly(L-lactide), poly(DL-lactide), poly(valerolactone), polyglycolide, polydioxanone, and their copolyesters. Examples of polycarbonates are poly(trimethylenecarbonate), poly(dimethyltrimethylenecarbonate), poly(hexamethylene carbonate).

The same result may be obtained with alternative, non-supramolecular polymers, if properties are carefully selected and material processed to ensure required surface characteristics. These polymers may comprise biodegradable or non-biodegradable polyesters, polyurethanes, polycarbonates, poly(orthoesters), polyphosphoesters, polyanhydrides, polyphosphazenes, polyhydroxyalkanoates, polyvinylalcohol, polypropylenefumarate. Examples of polyesters are polycaprolactone, poly(L-lactide), poly(DL-lactide), poly(valerolactone), polyglycolide, polydioxanone, and their copolyesters. Examples of polycarbonates are poly(trimethylenecarbonate), poly(dimethyltrimethylenecarbonate), poly(hexamethylene carbonate).

What is claimed is:

1. A medical implant, comprising:
a leaflet of an engineered heart valve, wherein the leaflet has two electrospun layers which are attached only at the edges of the two electrospun layers allowing for maximum and independent movability of the one layer relative to the other layer in the leaflet.

2. The medical implant as set forth in claim 1, wherein the leaflet is porous and bioabsorbable making the leaflet capable to be absorbed and replaced by natural tissue due to ingrowth of cells and nutrients into pores.

3. The medical implant as set forth in claim 1, further comprising a middle layer in between the two electrospun layers, wherein the middle layer remains independently movable from the two electrospun layers.

4. The medical implant as set forth in claim 3, wherein the middle layer is (i) porous and bioabsorbable making the middle layer capable to be absorbed and replaced by natural tissue due to ingrowth of cells and nutrients into pores, (ii) an electrospun layer which porous and bioabsorbable making the middle layer capable to be absorbed and replaced by natural tissue due to ingrowth of cells and nutrients into pores, or (iii) not bioabsorbable and capable to be permeated with natural tissue due to ingrowth of cells and nutrients into pores.

5. The medical implant as set forth in claim 3, wherein the middle layer and the two electrospun layers are electrospun with the same material or different materials.

6. The medical implant as set forth in claim 3, wherein the each of the two electrospun layers are electrospun with the same material or different materials.

7. A medical implant, comprising:
a leaflet of an engineered heart valve having a free edge, wherein the leaflet has a single electrospun layer folded over itself to have a bi-layered design for the leaflet such that the free edge of the heart valve is the folded edge of the leaflet.

8. The medical implant as set forth in claim 7, wherein only the remaining un-folded edges of the electrospun layer are attached and incorporated to a base of the heart valve allowing for maximum and independent movability of the one layer relative to the other layer in the leaflet.

9. The medical implant as set forth in claim 7, wherein the leaflet is porous and bioabsorbable making the leaflet capable to be absorbed and replaced by natural tissue due to ingrowth of cells and nutrients into pores.

10. The medical implant as set forth in claim 7, further comprising a middle layer in between the bi-layered design, wherein the middle layer remains independently movable from the bi-layered design.

11. The medical implant as set forth in claim 10, wherein the middle layer is (i) porous and bioabsorbable making the middle layer capable to be absorbed and replaced by natural tissue due to ingrowth of cells and nutrients into pores, (ii) an electrospun layer which porous and bioabsorbable making the middle layer capable to be absorbed and replaced by natural tissue due to ingrowth of cells and nutrients into pores, or (iii) not bioabsorbable and capable to be permeated with natural tissue due to ingrowth of cells and nutrients into pores.

12. The medical implant as set forth in claim 10, wherein the middle layer and the bi-layered design are electrospun with the same material or different materials.

13. A medical implant, comprising:
a leaflet of an engineered heart valve, wherein the leaflet has two electrospun outer layers and a middle layer, wherein the middle layer is laminated between the two outer layers and not independently movable from the two electrospun outer layers.

14. The medical implant as set forth in claim 13, wherein the middle layer is (i) porous and bioabsorbable making the middle layer capable to be absorbed and replaced by natural tissue due to ingrowth of cells and nutrients into pores, (ii) an electrospun layer which porous and bioabsorbable making the middle layer capable to be absorbed and replaced by natural tissue due to ingrowth of cells and nutrients into pores, or (iii) not bioabsorbable and capable to be permeated with natural tissue due to ingrowth of cells and nutrients into pores.

15. The medical implant as set forth in claim 13, wherein the middle layer and the two electrospun outer layers are electrospun with the same material or different materials.

16. The medical implant as set forth in claim 13, wherein the each of the two electrospun layers are electrospun with the same material or different materials.
